(19) United States
(12) Patent Application Publication
(10) Pub. No.: US 2024/0193417 A1
(43) Pub. Date: Jun. 13, 2024

(54) APPARATUS FOR NON-DETERMINISTIC FUTURE STATE PREDICTION USING TIME SERIES DATA AND OPERATION METHOD THEREOF

(71) Applicant: ELECTRONICS AND TELECOMMUNICATIONS RESEARCH INSTITUTE, Daejeon (KR)

(72) Inventors: Hwin Dol PARK, Daejeon (KR); Do Hyeun KIM, Daejeon (KR); Jae Hun CHOI, Daejeon (KR)

(21) Appl. No.: 18/504,214

(22) Filed: Nov. 8, 2023

(30) Foreign Application Priority Data
Dec. 13, 2022 (KR) 10-2022-0174047

(51) Int. Cl. G06N 3/08

(52) U.S. Cl. CPC G06N 3/08

(57) ABSTRACT

Disclosed is an apparatus, which includes a preprocessor that generates raw data, generates preprocessed time series data, and generates preprocessed learning data, and a learner that receives the preprocessed learning data as input data and trains a prediction model such that the similarity between a first future state predicted using the input data and a second future state predicted using data included in the same cluster as the input data increases and such that the similarity between the first future state and a third future state predicted using data included in a different cluster from the input data decreases, and the prediction model is a machine learning model for predicting a future state of the time series data at an arbitrary time point.

### CROSS-REFERENCE TO RELATED APPLICATION

This application claims priority under 35 U.S.C. § 119 to Korean Patent Application No. 10-2022-0174047 filed on Dec. 13, 2022, in the Korean Intellectual Property Office, the disclosures of which are incorporated by reference herein in their entireties.

### BACKGROUND

### 1. Field of the Invention

Embodiments of the present disclosure described herein relate to processing of time series data, and more particularly, relate to an apparatus for a non-deterministic future state prediction using time series data and operation method thereof.

### 2. Description of Related Art

Technologies for predicting future states using time series data recorded sequentially over time include an AR (autoregressive) model, a MA (moving average) model, an ARMA (autoregressive and moving average) model, an ARIMA (autoregressive integrated moving average), a CNN (convolutional neural network), an RNN (recurrent neural network), etc. In particular, the RNN is a representative time series data processing method, and is a technology that receives time series data sequentially and predicts a future time interval by simultaneously considering past and current time intervals. In the medical field, the RNN may be used to plan treatment in a way that reduces the probability of disease occurring.

However, when simply modeling the time series data in chronological order, it becomes difficult to reflect information such as the period of disease or the period of drug administration. To improve this, studies are also conducted to predict the risk of disease using an attention network method that may consider the period of suffering from a disease or the period of drug administration by adding period sequence data along with the time series data.

However, since most patients are likely to already be suffering from a disease, it may be of little use for medical professionals to predict the risk of a disease when planning a treatment for patients, and it may be difficult to determine at what point the predicted disease will develop using only period sequence information indicating the period of time the patient suffered from the disease. Furthermore, since the effect of a drug may be different for each patient and resistance to a specific drug may develop, a disease risk prediction does not accurately reflect the different treatment responsiveness of each patient. Therefore, in addition to predicting the risk of disease, research is required on methods to predict various patient conditions that may change depending on treatment.

### SUMMARY

Embodiments of the present disclosure provide an apparatus for predicting non-deterministic future states using time series data to predict treatment results reflecting different treatment responsiveness for each patient, and a method of operating the same.

According to an embodiment of the present disclosure, an apparatus includes a preprocessor that generates raw data by removing or replacing an outlier and a missing value in time series data, generates preprocessed time series data by converting the raw data into one integrated data, and generates preprocessed learning data by clustering the preprocessed time series data depending on a similarity, and a learner that receives each data of the preprocessed learning data as input data and trains a prediction model such that the similarity between a first future state predicted using the input data and a second future state predicted using data included in the same cluster as the input data increases and such that the similarity between the first future state and a third future state predicted using data included in a different cluster from the input data decreases, and the prediction model is a machine learning model for predicting a future state of the time series data at an arbitrary time point.

According to an embodiment of the present disclosure, an apparatus includes a preprocessor that generates raw data by removing or replacing an outlier and a missing value of time series data and converts the raw data into one integrated data to generate preprocessed time series data, and a predictor that generates a prediction result corresponding to a future state at an arbitrary next time point in time through a prediction model, based on the preprocessed time series data, event data indicating an additional state not included in the preprocessed time series data, and next time point data indicating the arbitrary next time point, and the prediction model is a machine learning model for predicting a future state of the time series data at an arbitrary time point.

According to an embodiment of the present disclosure, a method of operating an apparatus for non-deterministic future state prediction using time series data includes generating raw data by removing or replacing an outlier and a missing value in time series data, generating preprocessed time series data by converting the raw data into one integrated data, generating preprocessed learning data by clustering the preprocessed time series data depending on a similarity, receiving each data of the preprocessed learning data as input data, generating group learning data by grouping the input data, similar data included in the same cluster as the input data, and dissimilar data included in a different cluster from the input data, training a prediction model such that the similarity between a first future state predicted using the input data and a second future state predicted using the similar data increases and such that the similarity between the first future state and a third future state predicted using the dissimilar data decreases, and generating a prediction result corresponding to a future state at an arbitrary next time point of the time series data received from a user through the prediction model.

### BRIEF DESCRIPTION OF THE DRAWINGS

The above and other objects and features of the present disclosure will become apparent by describing in detail embodiments thereof with reference to the accompanying drawings.

FIG. 1 is a block diagram illustrating a configuration of an apparatus for a non-deterministic future state prediction, according to an embodiment of the present disclosure.

FIG. 2 is a block diagram illustrating a configuration of a preprocessor of FIG. 1.

FIG. 3 illustrates an example of an operation of a time series feature converter of FIG. 2.

FIG. 4 illustrates an example of an operation of a similar state classifier of FIG. 2.

FIG. 5 is a block diagram illustrating a configuration of a learner of FIG. 1.

FIG. 6 illustrates an example of an operation of a similar feature generator of FIG. 5.

FIG. 7 illustrates an example of an operation of a multi-state calculator of FIG. 5.

FIG. 8 illustrates an example of an operation of a multi-state probability estimator of FIG. 5.

FIG. 9 illustrates an example of an operation of a distance calculator of FIG. 5.

FIG. 10 is a block diagram illustrating a configuration of the predictor of FIG. 1.

FIG. 11 illustrates an example of an operation of a next time point reflector of FIG. 10.

FIG. 12 is a block diagram illustrating a configuration of an apparatus for a non-deterministic future state prediction that includes only a preprocessor and a learner.

FIG. 13 is a block diagram illustrating a configuration of an apparatus for a non-deterministic future state prediction that includes only a preprocessor and a predictor.

FIG. 14 is a flowchart illustrating a method of operating an apparatus for a non-deterministic future state prediction, according to an embodiment of the present disclosure.

### DETAILED DESCRIPTION

Hereinafter, embodiments of the present disclosure will be described in detail and clearly to such an extent that an ordinary one in the art easily implements the present disclosure.

Components that are described in the detailed description with reference to the terms “unit”, “module”, “block”, “˜er or ˜or”, etc. and function blocks illustrated in drawings will be implemented with software, hardware, or a combination thereof. For example, the software may be a machine code, firmware, an embedded code, and application software. For example, the hardware may include an electrical circuit, an electronic circuit, a processor, a computer, an integrated circuit, integrated circuit cores, a pressure sensor, an inertial sensor, a microelectromechanical system (MEMS), a passive element, or a combination thereof.

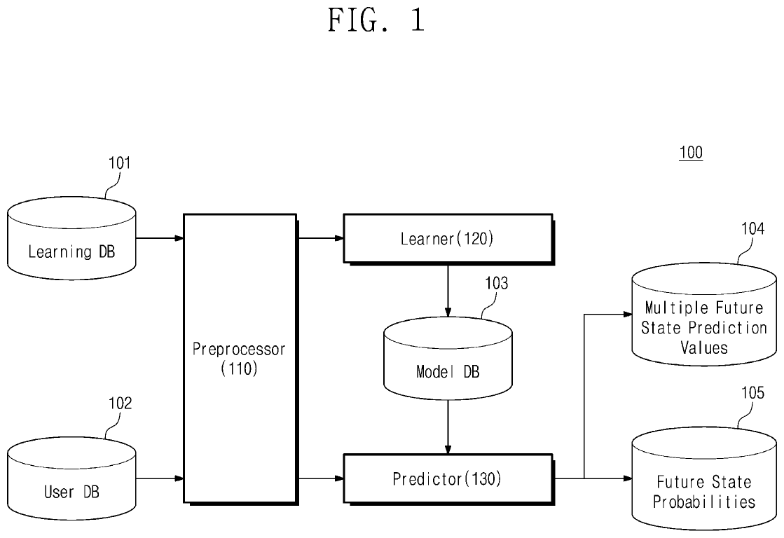

FIG. 1 is a block diagram illustrating a configuration of an apparatus 100 for a non-deterministic future state prediction, according to an embodiment of the present disclosure. The apparatus 100 of FIG. 1 may preprocess time series data, may train a prediction model to predict a future state based on the preprocessed time series data, and may use the predicted model to predict the future state at an arbitrary time point desired by a user. Referring to FIG. 1, the apparatus 100 may include a preprocessor 110, a learner 120, and a predictor 130.

The time series data may be a set of data recorded over time and in temporal order. The time series data may include at least one feature corresponding to each of a plurality of times listed in time series. For example, the time series data may include time series medical data indicating user's health states generated by diagnosis, treatment, or medication prescription at a medical institution, such as an electronic medical record (EMR). Hereinafter, for clarity of description, it is assumed that the time series data of the present disclosure is time series medical data such as the EMR, but the types of time series data are not limited thereto.

For example, the preprocessor 110, the learner 120, and the predictor 130 may be implemented as hardware, firmware, software, or a combination thereof. For example, the software (or firmware) may be loaded onto a memory (not illustrated) included in the apparatus 100 and may be executed by a processor (not illustrated). For example, the preprocessor 110, the learner 120, and the predictor 130 may be implemented with hardware such as a dedicated logic circuit such as a Field Programmable Gate Array (FPGA) or an Application Specific Integrated Circuit (ASIC), but the present disclosure is not limited thereto.

The preprocessor 110 may receive the time series data and may preprocess the time series data such that the time series data may be used for learning and prediction. In detail, the preprocessor 110 may remove or replace an outlier or a missing value present in time series data, may convert them into one integrated data form (e.g., a matrix) that may be used for learning and prediction, and may cluster the converted data depending on similarity.

The preprocessor 110 may preprocess the time series data received from a learning database 101 and may provide the preprocessed time series data to the learner 120, and the preprocessor 110 may preprocess the time series data received from a user database 102 and may provide the preprocessed time series data to the predictor 130. The learning database 101 and the user database 102 may be implemented on a server or storage medium external or internal to the apparatus 100. In the learning database 101 and the user database 102, data may be managed in time series, grouped, and stored.

The learner 120 may train a prediction model to predict the future state at an arbitrary time point based on the preprocessed time series data. The prediction model may include a time series analysis model (i.e., a machine learning model) for predicting the future state at an arbitrary time point desired by a user by analyzing the preprocessed time series data. For example, the prediction model may be implemented through neural networks such as a Convolutional Neural Network (CNN), a Recurrent Neural Network (RNN), a Bayesian Neural Network (BNN), etc.

In detail, the learner 120 may group data with similar and dissimilar specific features for each of the time series data received for learning, and may calculate a vector for predicting the future state of the time series data. Subsequently, the learner 120 may predict possible future states of the received time series data using the calculated vector. In more detail, the learner 120 may train a prediction model such that the similarity between the future state predicted using the received time series data and the future state predicted using similar data (data included in the same cluster) increases, and such that the similarity between the future state predicted using the received time series data and the future state predicted using dissimilar data (data included in another cluster) decreases. The trained prediction model may be stored and managed through a model database 103.

The predictor 130 may generate a prediction result corresponding to the future state at an arbitrary next time point with respect to the preprocessed time series data. The predictor 130 may analyze the preprocessed time series data based on the prediction model trained in the learner 120. To this end, the predictor 130 may receive a prediction model from the model database 103.

In detail, the predictor 130 may use the prediction model to predict future states (e.g., in the case of time series medical data, state information such as patient's red blood cells, calcium, uric acid levels, etc. or values representing treatment information such as whether to take antibiotics) of the received time series data at an arbitrary next time point, then, may generate multiple future state prediction values 104, and may generate future state probabilities 105 by calculating the probability corresponding to each of the predicted future states. The multiple future state prediction values 104 and the future state probabilities 105 may be stored and managed as one database.

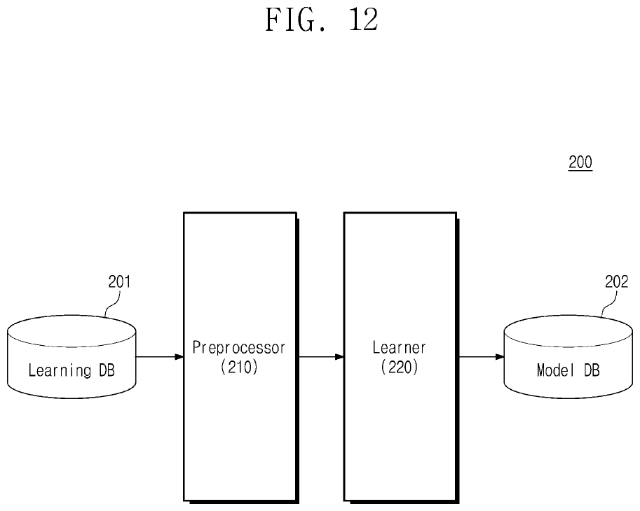

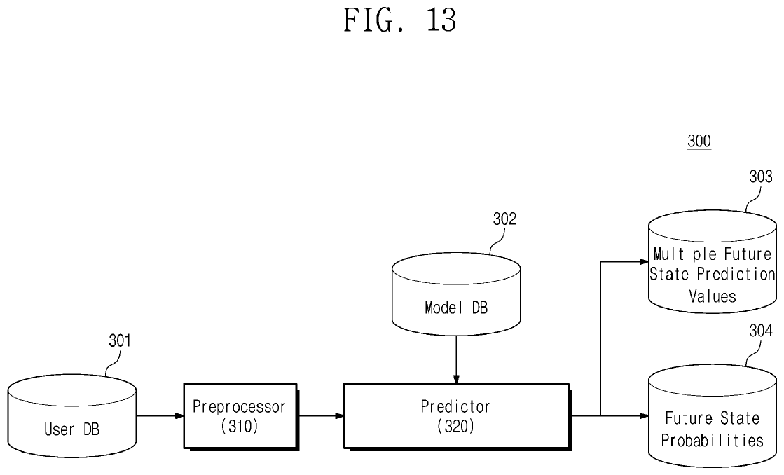

Meanwhile, according to an embodiment of the present disclosure, the preprocessor 110, the learner 120, and the predictor 130 may be included in one apparatus 100, but in some cases, only the learning operation of the prediction model may be performed using only the preprocessor and the learner, or only the prediction operation using the prediction model may be performed using only the preprocessor and the predictor. This will be described later with reference to FIGS. 12 and 13.

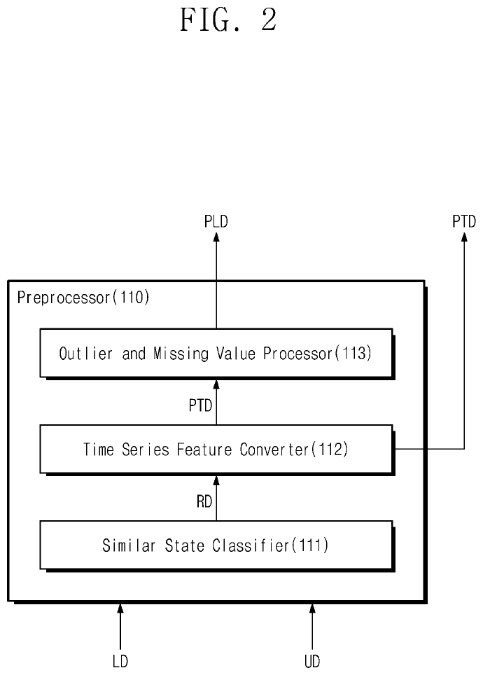

FIG. 2 is a block diagram illustrating a configuration of the preprocessor 110 of FIG. 1. Referring to FIG. 2, the preprocessor 110 may include an outlier and missing value processor 111, a time series feature converter 112, and a similar state classifier 113. As described with reference to FIG. 1, the preprocessor 110 may receive learning data LD for training a prediction model from the learning database 101, and may receive user data UD for predicting the future state at a desired time point from the user database 102. For example, both learning data LD and user data UD may be time series medical data.

The outlier and missing value processor 111 may remove or replace outliers or missing values present in the learning data LD and the user data UD. For example, in the case of time series medical data, blood pressure or body temperature recorded as being higher than the actual value may correspond to the outliers, and items that are not recorded may correspond to the missing values. The outlier and missing value processor 111 may generate raw data RD by removing or replacing outliers or missing values with reference to previous values or average values of the time series data. The outlier and missing value processor 111 may provide the raw data RD to the time series feature converter 112.

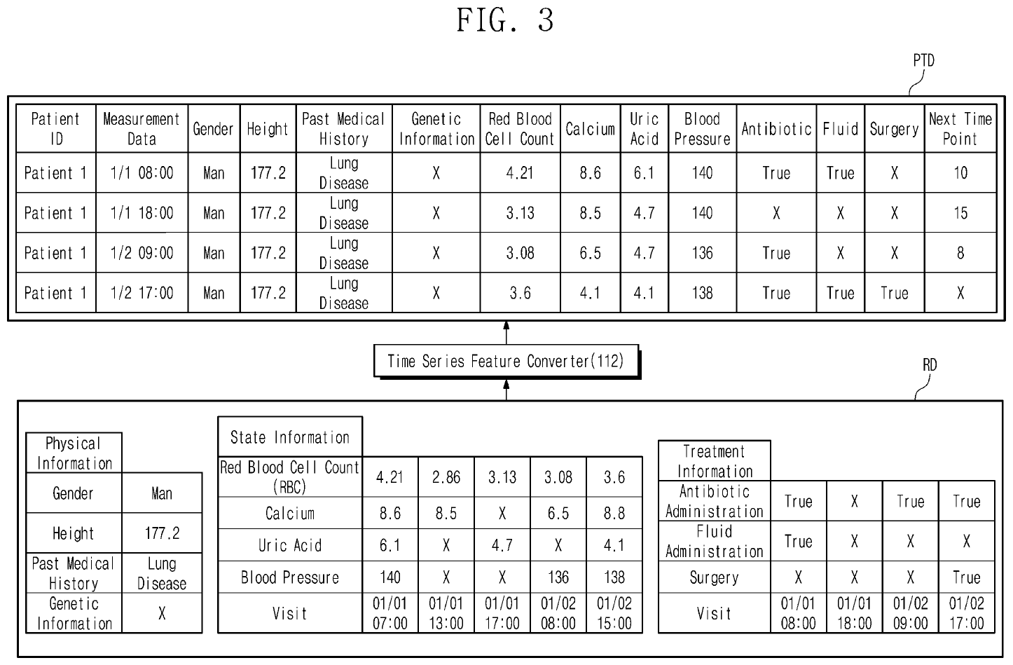

The time series feature converter 112 may convert the raw data RD into a single integrated data form (e.g., a matrix) that may be used for learning and prediction, and may output the converted raw data as preprocessed time series data PTD. In this case, the preprocessed time series data PTD based on learning data LD may be provided to the similar state classifier 113 for learning, and the preprocessed time series data PTD based on user data UD may be provided to the predictor 130 for prediction. An operation of the time series feature converter 112 will be described in more detail with reference to FIG. 3.

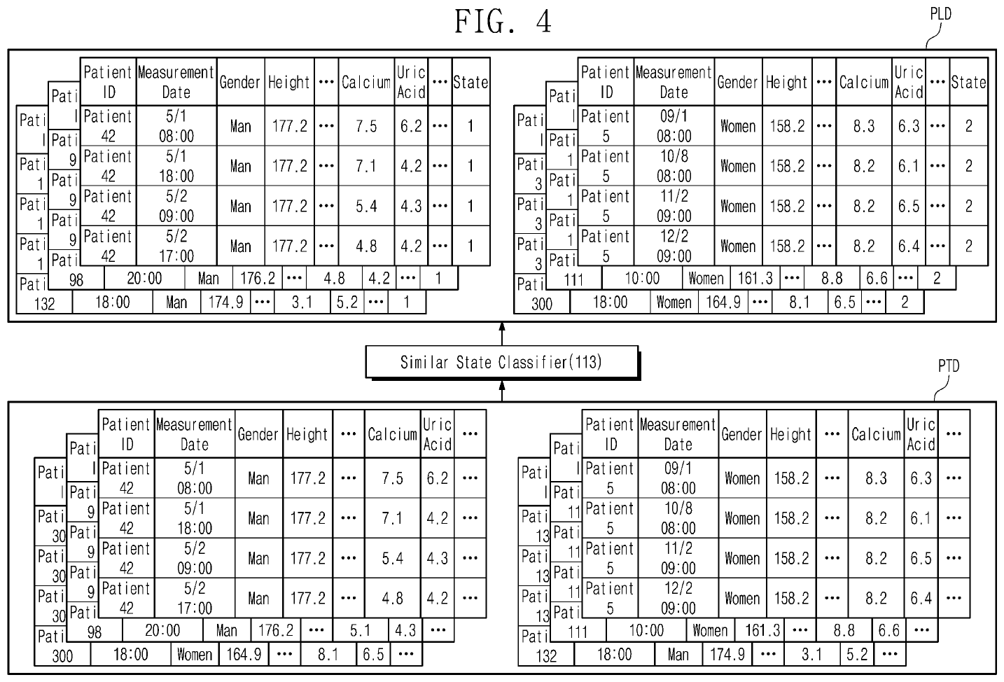

The similar state classifier 113 may cluster the preprocessed time series data PTD depending on similarity. For example, the similar state classifier 113 may apply a clustering algorithm such as K-means clustering to the generated preprocessed time series data PTD to generate time series data of patients with similar states as one cluster. The similar state classifier 113 may output the clusters generated in this way as the preprocessed learning data PLD and may provide the clusters to the learner 120. An operation of the similar state classifier 113 will be described in more detail with reference to FIG. 4.

FIG. 3 illustrates an example of an operation of the time series feature converter 112 of FIG. 2. The time series feature converter 112 may convert the raw data RD received from the outlier and missing value processor 111 into one integrated matrix form. For example, as illustrated in FIG. 3, the raw data RD may include physical information as a single record, patient's state information recorded in chronological order, and patient's treatment information recorded in chronological order. The time series feature converter 112 may convert them into a matrix form arranged in chronological order (e.g., in order of 1/1 08:00, 1/1 18:00, 1/2 09:00, and 1/2 17:00), and may generate the converted matrix as the preprocessed time series data PTD.

When the preprocessed time series data PTD is generated, the time series feature converter 112 may generate a time interval (i.e., the time point at which the next data is to be recorded) between time points as the next time point. For example, in the preprocessed time series data PTD in FIG. 3, the next time points may be 10 hours (between 1/1 08:00 and 1/1 18:00), 15 hours (between 1/1 18:00 and 1/2), and 8 hours (between 1/2 09:00 and 1/2 17:00), respectively. There is no data corresponding to the next time point after 1/2 17:00, so it is indicated as X. As described with reference to FIG. 2, the preprocessed time series data PTD may be provided to the similar state classifier 113 or the predictor 130.

FIG. 4 illustrates an example of an operation of the similar state classifier 113 of FIG. 2. The similar state classifier 113 may cluster the preprocessed time series data PTD received from the time series feature generator 112 depending on similarity. Referring to FIG. 4, the preprocessed time series data PTD of patients with similar state information (e.g., patients with similar calcium and uric acid levels) may be generated as one cluster (e.g., a first cluster containing patients 42, 98, and 132 with state 1 or a second cluster containing patients 5, 111, and 300 with state 2). For example, the similar state classifier 113 may apply the K-means clustering algorithm to numerical values representing the patient's state information, but the present disclosure is not limited thereto. The generated clusters may be output as the preprocessed learning data PLD and may be provided to the learner 120.

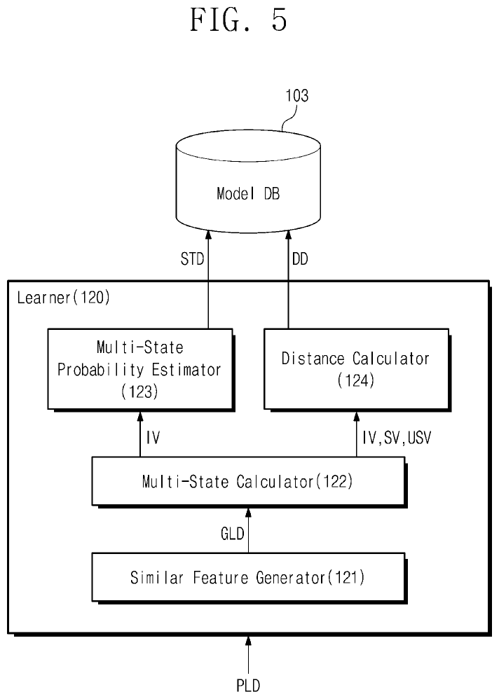

FIG. 5 is a block diagram illustrating a configuration of the learner 120 of FIG. 1. Referring to FIG. 5, the learner 120 may include a similar feature generator 121, a multi-state calculator 122, a multi-state probability estimator 123, and a distance calculator 124. The learner 120 may receive the preprocessed learning data PLD for training a prediction model from the similar state classifier 113 of the preprocessor 110.

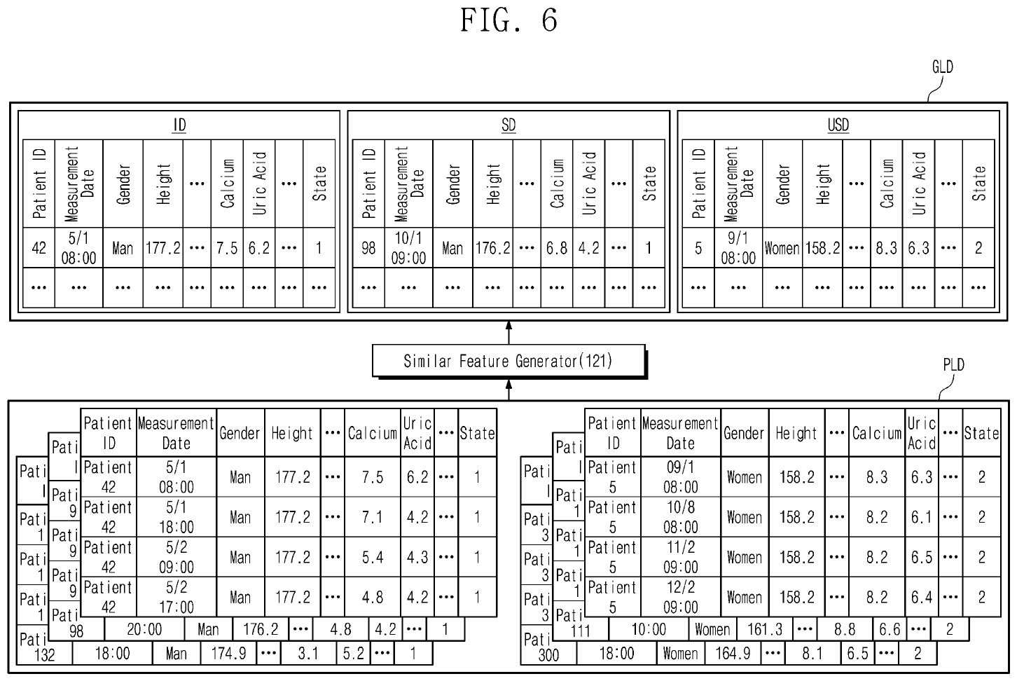

The similar feature generator 121 may group a matrix similar to (hereinafter referred to as similar data) input data and a matrix not similar to (hereinafter referred to as dissimilar data) the input data into one matrix for each of the matrices (hereinafter referred to as the input data) included in the preprocessed learning data PLD received from the similar state classifier 113 to group learning data GLD. Since there may be more than one similar data and dissimilar data corresponding to one input data, the group learning data GLD is not set to one, and may change flexibly according to the learning process of the prediction model. The similar feature generator 121 may provide the group learning data GLD to the multi-state calculator 122. An operation of the similar feature generator 121 will be described in more detail with reference to FIG. 6.

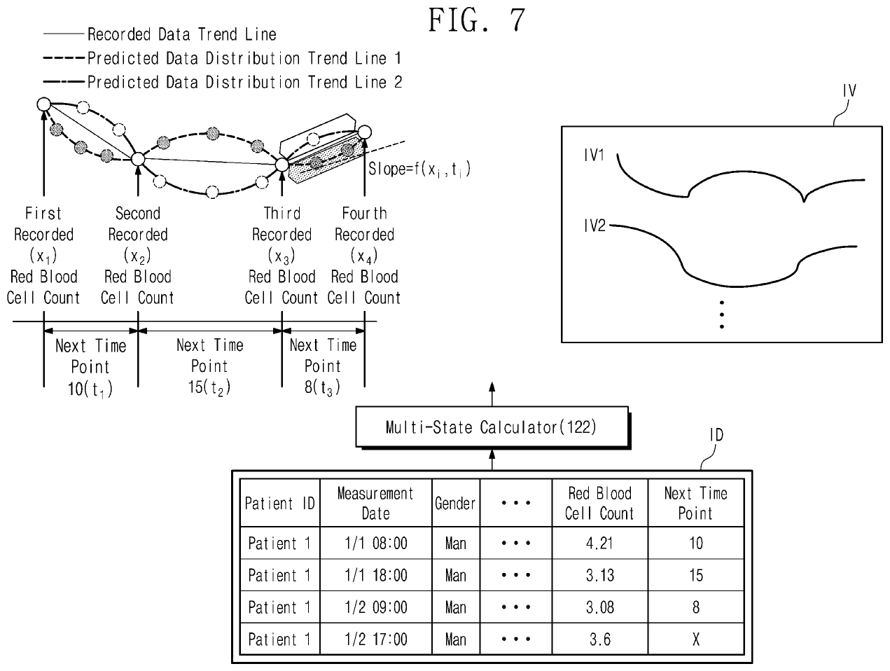

The multi-state calculator 122 may use the group learning data GLD provided from the similar feature generator 121 to calculate latent vectors representing changes over time in each of the input data, the similar data, and the dissimilar data. The latent vector may be defined as a vector space that estimates the value of the next state by reflecting the features of the model. In detail, the multi-state calculator 122 may generate input latent vectors IV reflecting the time series features of the input data, similar latent vectors SV reflecting the time series features of the similar data, and dissimilar latent vectors USV reflecting the time series features of dissimilar data, for each of the matrices of the group learning data GLD. The multi-state calculator 122 may provide the input latent vectors IV to the multi-state probability estimator 123 and the distance calculator 124, and may provide the similar latent vectors SV and the dissimilar latent vectors USV to the distance calculator 124. An operation of the multi-state calculator 122 will be described in more detail with reference to FIG. 7.

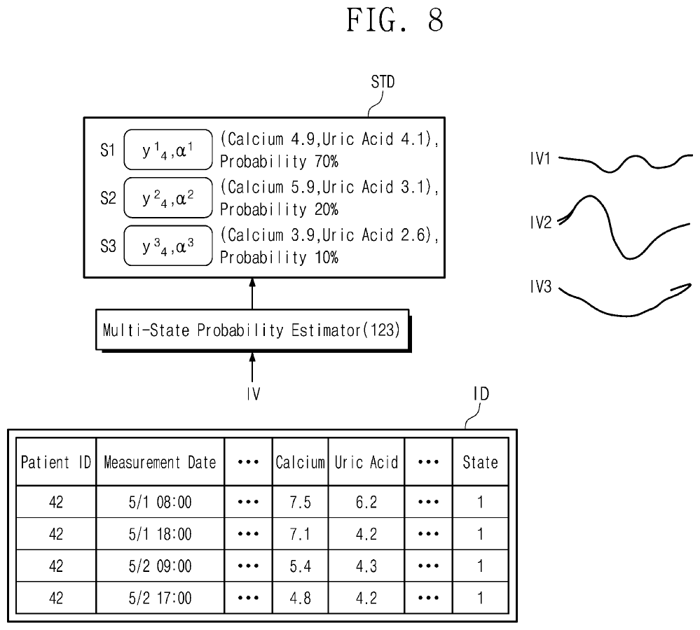

The multi-state probability estimator 123 may use the input latent vectors IV calculated in the multi-state calculator 122 to predict values (e.g., the patient's calcium and uric acid levels, etc.) associated with possible next states of the input data of the group learning data GLD and the probability of reaching those states. The multi-state probability estimator 123 may output the values associated with the next states and the probabilities as state data STD. Through this, the multi-state probability estimator 123 may train a prediction model to increase the probability of reaching the next state with values closest to the actual state among a plurality of possible next states. An operation of the multi-state probability estimator 123 will be described in more detail with reference to FIG. 8.

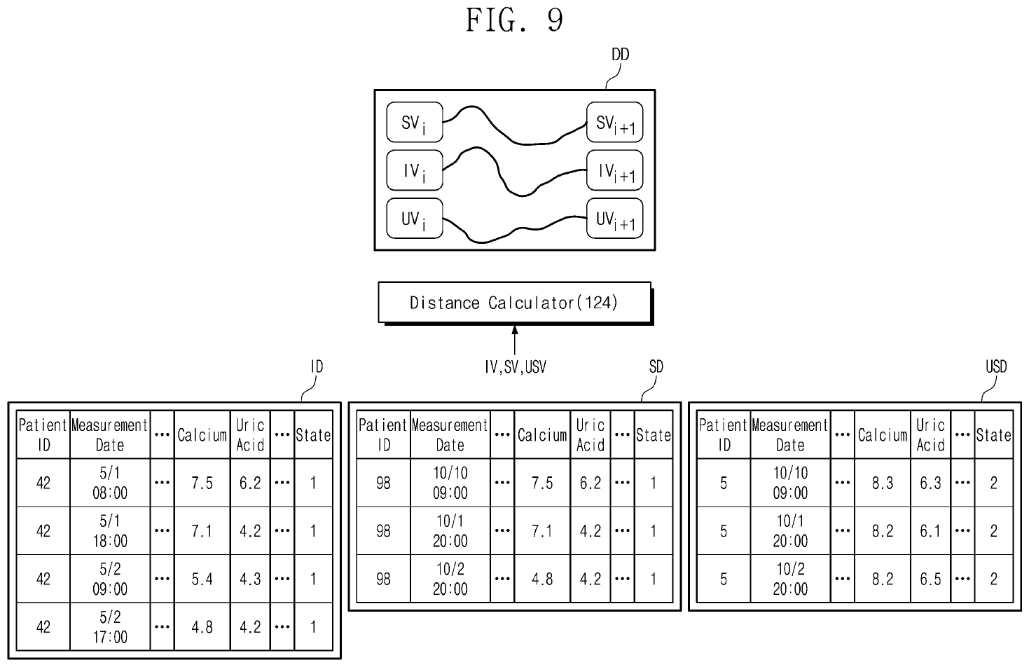

The distance calculator 124 may use the input latent vectors IV, the similar latent vectors SV, and the dissimilar latent vectors USV calculated in the multi-state calculator 122 to calculate a similarity distance (hereinafter referred to as similarity distance) between the next state of the input data and the next state of similar data, and a similarity distance (hereinafter referred to as dissimilarity distance) between the next state of input data and the next state of dissimilar data, for each matrix of the group learning data GLD. The distance calculator 124 may output such similarity distances as distance data DD. Through this, the distance calculator 124 may train a prediction model such that the similarity distance becomes closer and the dissimilarity distance becomes farther. An operation of the distance calculator 124 will be described in more detail with reference to FIG. 9.

FIG. 6 illustrates an example of an operation of the similar feature generator 121 of FIG. 5. The similar feature generator 121 may group the similar data SD and the dissimilar data USD corresponding to each input data ID included in the preprocessed learning data PLD into one matrix to generate the group learning data GLD. For example, in the preprocessed learning data PLD, a matrix belonging to the same cluster as the input data ID may be determined as the similar data SD, and a matrix belonging to a different cluster may be determined as the dissimilar data USD. The similar feature generator 121 may provide the group learning data GLD to the multi-state calculator 122.

As described with reference to FIG. 4, one cluster may include a plurality of matrices having the same state. Accordingly, one input data ID may include one or more corresponding similar data SD and one or more corresponding dissimilar data USD. Referring to FIG. 6, the first cluster of the preprocessed learning data PLD includes the matrices of patient 42, patient 98, and patient 132, and the second cluster of the preprocessed learning data PLD includes the matrices of patient 5, patient 111, and patient 300. Therefore, based on patient 42, the matrices of patient 98 and patient 132 belonging to the same cluster may be determined as the similar data SD, and the matrices of patient 5, patient 111, and patient 300 belonging to a different cluster may be determined as the dissimilar data USD.

Referring to the group learning data GLD illustrated in FIG. 6, for the matrix of patient 42, which is the input data ID, the matrix of patient 98 is illustrated as the similar data SD and the matrix of patient 5 is illustrated as the dissimilar data USD, but the group learning data GLD is not limited thereto, and during the learning process of the prediction model, the similar data SD may be changed to the matrix of patient 132, and the dissimilar data USD may be changed to the matrix of patient 111. In detail, the similar feature generator 121 may change similar data to other similar data for the same input data ID, or may change dissimilar data to other dissimilar data, and may continuously generate the group learning data GLD, so that the group learning data GLD may change flexibly during the learning process of the prediction model. As a result, efficient learning of a prediction model may be possible even when the amount of data is insufficient and the imbalance (e.g., the imbalance between healthy patient data and patient data suffering from a rare disease)between the data.

FIG. 7 illustrates an example of an operation of the multi-state calculator 122 of FIG. 5. The multi-state calculator 122 may generate the input latent vectors IV, the similar latent vectors SV, and the dissimilar latent vectors USV representing trends over time for each of the input data ID, the similar data SD, and the dissimilar data USD, based on the group learning data GLD. For clear description, FIG. 7 illustrates the operation of generating the input latent vectors IV corresponding to the input data ID, and an operation of generating the similar latent vectors SV and the dissimilar latent vectors USV is also the same as the operation.

For example, referring to the matrix of patient 1 illustrated as input data ID in FIG. 7, the red blood cell count changes to 4.21, 3.13, 3.08, and 3.6 depending on the measurement date. For training of the prediction model, the multi-state calculator 122 may calculate the input latent vectors IV to estimate the last record (3.6) based on the first three records (4.21, 3.13, and 3.08). Based on the change in red blood cell count between 1/2 09:00 and 1/2 17:00, trend line 1 predicts that the red blood cell count will rise gently and then gradually rise rapidly to reach 3.6, and trend line 2 predicts that the red blood cell count will rise sharply and then gradually rise gently to reach 3.6. This is because changes in the state may appear differently depending on the patient even if the same treatment or drug is administered.

In addition, looking at the trend line between 1/2 09:00 and 1/2 17:00, the trend line 1 illustrates an area indicated by gray shading, and the trend line 2 illustrates an area indicated by a solid black. This means that even if it illustrates the same change trend over time, the trend line may appear differently within the displayed area due to random noise. In detail, the latent vector may be expressed as a trend line (e.g., predicted data distribution trend lines 1 and 2 in FIG. 7) indicating a change trend over time, but trend lines with similar change trends may appear in various ways according to an influenced (e.g., the solid black line or gray shaded areas illustrated in FIG. 7) of random noise.

In other words, the multi-state calculator 122 of the present disclosure may calculate a plurality of latent vectors for predicting multiple states by reflecting the influence of time series features and random noise with respect to the input data ID, the similar data SD, and the dissimilar data USD. For example, the multi-state calculator 122 may calculate the latent vector through Equation 1 below.

xⁿᵢ₊₁=xᵢ+∫fₙ(xᵢ,tᵢ)dt+∫gₙ(xᵢ,tᵢ)dN [Equation 1]

Where, xᵢ represents an i-th record, tᵢ represents the next time point of the i-th time point, xⁿᵢ₊₁ is a value representing the (i+1)-th record (i.e., values related to the next states) to be predicted, and the i-th record may include multiple values (i.e., multiple states) such as x¹ᵢ₊₁, x²ᵢ₊₁, . . . xⁿᵢ₊₁ depending on the latent vector being calculated. The fₙ(xᵢ, tᵢ) is a change rate estimation function, by taking xᵢ and tᵢ as input and integrating (∫fₙ(xᵢ,tᵢ)dt) over time, the amount of change in the record until the next time point may be estimated. The gₙ(xᵢ, tᵢ) is a diffusion degree estimation function, by taking xᵢ and tᵢ as input and integrating (∫gₙ(xᵢ,tᵢ)dN) over the random noise, the influence of random noise may be estimated until the next time point. By integrating the diffusion degree estimation function in this way, instability due to random noise (e.g., noise caused by outliers that are not removed) may be corrected, and the overfitting problem of the prediction model may also be solved.

The multi-state calculator 122 may include a machine learning model (e.g., a deep learning network) that may calculate integral values using a random rate of change estimation function and a diffusion degree estimation function. For example, the deep learning network may be implemented as any one of a CNN, an RNN, or a BNN, but the present disclosure is not limited thereto. The machine learning model of the multi-state calculator 122 may derive a rate of change estimation function that may correctly estimate the actual value (e.g., 3.6 red blood cell count at 1/2 17:00) and a diffusion estimation degree function by repeatedly calculating Equation 1 above.

The set of xⁿᵢ₊₁ calculated by adding the change rate and diffusion degree estimated for the i-th record xᵢ may configure one latent vector, and the multi-state calculator 122 may calculate the plurality of input latent vectors IV, similar latent vectors SV, and dissimilar latent vectors USV. As illustrated in FIG. 7, the input latent vectors IV may include different vectors (e.g., IV1, IV2) and, although not illustrated, the similar latent vectors SV and the dissimilar latent vectors USV may likewise contain different vectors. The multi-state calculator 122 may provide the calculated vectors IV, SV, and USV to the multi-state probability estimator 123 and the distance calculator 124.

FIG. 8 illustrates an example of an operation of the multi-state probability estimator 123 of FIG. 5. The multi-state probability estimator 123 may predict values (e.g., patient's calcium, uric acid levels, etc.) associated with the possible next states of the input data ID through each of the input latent vectors IV and the possibilities of reaching those states, and may output them as the state data STD. For example, the multi-state probability estimator 123 may include a fully connected layer for outputting the state data STD. The fully connected layer may convert values associated with predicted states into corresponding probabilities (e.g., using a softmax function). However, the present disclosure is not limited thereto, and depending on the implementation method, other types of layers other than the fully connected layer and other functions other than the softmax function may also be used.

The multi-state probability estimator 123 may train a prediction model using a loss function L based on the values associated with the next states and the probability (i.e., the state data STD) corresponding to each of the states, as illustrated in Equation 2 below.

L(y^{real},y,α)=−minpos(|y^{real}−y|)log(α) [Equation 2]

Where, y^{real} represents the value associated with the actual next state, y represents the value associated with the next state predicted by the input latent vector IV, α represents the probability (percentage) of reaching the predicted value, and minpos is a function that calculates the position of the minimum value. The y^{real}−y represents the error between the actual value and the predicted value, and the loss function is calculated smaller as the error is smaller and the probability is larger.

Referring to FIG. 8, it is assumed that the state of 5/2 17:00 is predicted in the matrix of patient 42 using the input latent vectors IV1, IV2, and IV3. The actual value y^{real} is calcium 4.8 and uric acid 4.2, a value y¹₄ of a first state S1 predicted by a first input latent vector IV1 is calcium 4.9 and uric acid 4.1, a value y²₄ of a second state S2 predicted by a second input latent vector IV2 is calcium 5.9 and uric acid 3.1, and a value y³₄ of a third state S3 predicted by a third input latent vector IV3 is calcium 3.9 and uric acid 2.6. In this case, the probabilities of reaching each state α¹, α², and α³ are calculated to be 70%, 20%, and 10%, respectively.

In this case, since the error between the actual calcium and uric acid levels and the calcium and uric acid levels in the first state S1 is the smallest as 0.1, the value of the loss function calculated by Equation 2 may also be calculated as the smallest. Therefore, in this case, the multi-state probability estimator 123 may train the prediction model by using the state data STD such that the probability of reaching the first state S1, which is closest to the actual state is close to 100%, and the probability of reaching the remaining states is close to 0%.

FIG. 9 illustrates an example of an operation of the distance calculator 124 of FIG. 5. The distance calculator 124 may calculate a similar distance SIMD, which is the similarity between a next state IVᵢ₊₁ of the input data and a next state SVᵢ₊₁ of the similar data, and a dissimilar distance USIMD, which is the similarity between the next state IVᵢ₊₁ of the input data and a next state USVᵢ₊₁ of the dissimilar data, based on the input latent vectors IV, the similar latent vectors SV, and the dissimilar latent vectors USV. The similar distance SIMD may be calculated as the distance between the input latent vector IV and the similar latent vector SV, and the dissimilar distance USIMD may be calculated as the distance between the input latent vector IV and the dissimilar latent vector USV. For example, the distance between vectors may be calculated using Euclidean distance or cosine similarity, but the present disclosure is not limited thereto.

The distance calculator 124 may train the prediction model using the loss function L based on the similar distance SIMD and the dissimilar distance USIMD (i.e., distance data DD) of the next state as illustrated in Equation 3 below.

L(IV,SV,USV)=max(d(IV−SV)−d(IV−USV)+C,0) [Equation 3]

Where, d(IV−SV) represents the similar distance SIMD, d(IV−USV) represents the dissimilar distance USIMD, and ‘C’ is a constant representing the minimum distance. The constant C may be pre-specified by the user, or may be set to the minimum value among the distances between states of the matrix included in the same cluster. The loss function is smaller as the value of d(IV−SV) is smaller (i.e., as the similar distance SIMD is closer) and the value of d(IV−USV) is larger (i.e., as the dissimilar distance USIMD is farther), and ultimately, when the value of d(IV−SV)+C is less than the value of d(IV−USV), the loss function becomes 0. In this way, the distance calculator 124 may train (e.g., changing a weight of the prediction model) the prediction model such that the input latent vector IV and the similar latent vector SV become closer, and the input latent vector IV and the dissimilar latent vector USV become farther.

For example, referring to FIG. 9, the matrix of patient 42 is illustrated as input data ID, the matrix of patient 98 is illustrated as similar data SD, and the matrix of patient 5 is illustrated as dissimilar data USD. In addition, it may be seen that the calcium and uric acid levels at 5/2 17:00 of patient 42 and the calcium and uric acid levels at 10/2 20:00 of patient 98 are similar to each other, and the calcium and uric acid levels at 5/2 17:00 of patient 42 and the calcium and uric acid levels at 10/2 20:00 of patient 5 are not similar to each other. In this case, the distance calculator 124 may train the prediction model such that the distance between the input latent vector corresponding to 5/2 17:00 of patient 42 and the similar latent vector corresponding to 10/2 20:00 of patient 98 is closer, and the distance between the input latent vector corresponding to 5/2 17:00 of patient 42 and the dissimilar latent vector corresponding to 10/2 20:00 of patient 5 is farther.

Furthermore, by calculating the distance data DD including the similar distance SIMD and the dissimilar distance USIMD in the latent vector dimension, the distance calculator 124 may also easily calculate the similarity between time series data with different numbers of time points. Through this, the distance calculator 124 may train a prediction model using time series data with different numbers of time points.

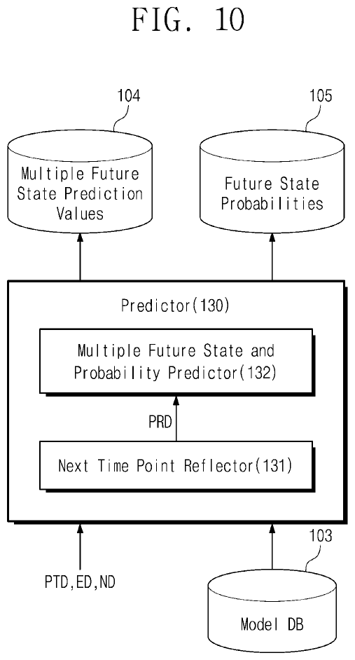

FIG. 10 is a block diagram illustrating a configuration of the predictor 130 of FIG. 1. Referring to FIG. 10, the predictor 130 may include a next time point reflector 131 and a multiple future state and probability predictor 132. The predictor 130 may receive the preprocessed time series data PTD for prediction from the time series feature generator 112 of the preprocessor 110. Furthermore, the predictor 130 may receive event data ED related to preprocessed time series data PTD and next time point data ND indicating an arbitrary next time point to be predicted. For example, the event data ED may represent additional states that are not included in the preprocessed time series data PTD, and may be omitted when no states are specifically added. The predictor 130 may receive the prediction model trained by the learner 120 from the model database 103 and may generate a prediction result corresponding to the future state of the preprocessed time series data PTD at an arbitrary next time point.

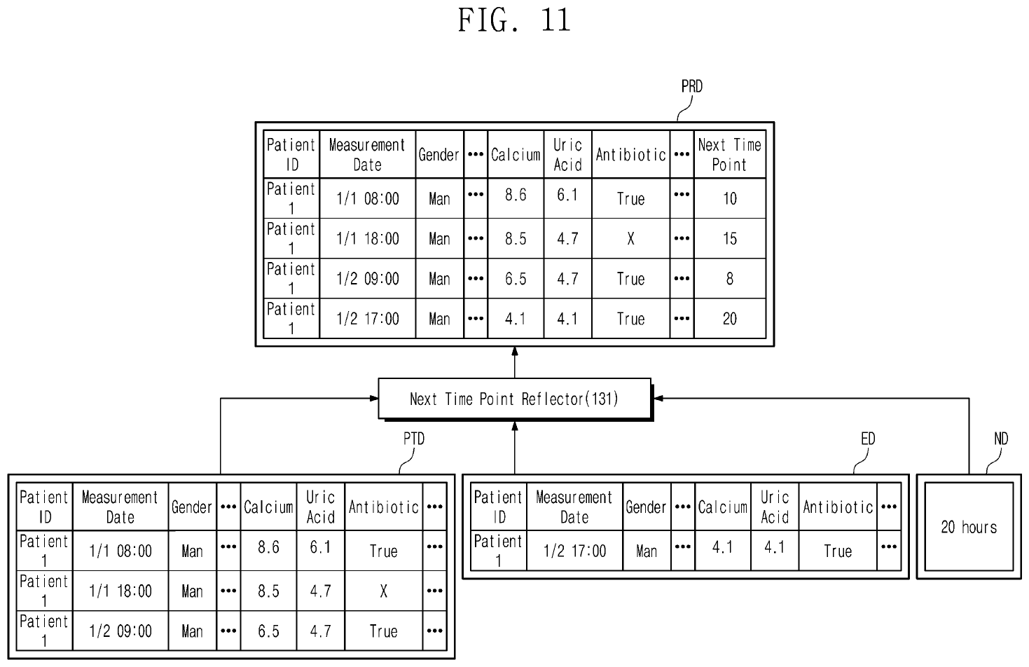

The next time point reflector 131 may generate prediction data PRD based on preprocessed time series data PTD, the event data ED, and the next time point data ND. For example, the next time point reflector 131 may add the event data ED indicating the current additional state to the preprocessed time series data PTD, and may set the next time point corresponding to the added event data ED to the next time point data ND. The prediction data PRD generated in this way may be provided to the multiple future state and probability predictor 132 to predict the next state and probability by the prediction model. An operation of the next time point reflector 131 will be described in more detail with reference to FIG. 11.

The multiple future state and probability predictor 132 may apply the trained prediction model to the prediction data PRD provided from the next time point reflector 131 to output values (the multiple future state prediction values 104) related to possible future states and the probability (the future state probabilities 105) corresponding to each of the states. Through this, the user may identify changes that may occur at any next time point depending on the event. In other words, through the above-described operation, the predictor 130 according to an embodiment of the present disclosure may predict a non-deterministic future state at any next time point that may vary depending on additional events even if the same time series data is given.

FIG. 11 illustrates an example of the operation of the next time point reflector 131 of FIG. 10. The next time point reflector 131 may generate the prediction data PRD reflecting additional events and next time points based on the preprocessed time series data PTD based on the user data UD, the received event data ED, and the next time point data ND. For example, the event data ED may refer to events that a medical professional is planning for a specific patient. By receiving the event data ED in this way, it is possible to predict the patient's state when new treatment is performed based on past data. The next time point data ND may represent an arbitrary time point in the desired future state.

Referring to FIG. 11, the preprocessed time series data PTD is a matrix containing information on 1/1 08:00, 1/1 18:00, and 1/2 09:00 of patient 1, and the event data ED is a matrix containing information on 1/2 17:00 of patient 1, and the next time point data ND corresponds to 20 hours. In conclusion, the predicted data PRD may indicate a matrix for predicting the results 20 hours later when performing a new treatment (when antibiotics are administered at 1/2 17:00) based on historical data (information on 1/1 08:00, 1/1 18:00, and 1/2 09:00). The next time point reflector 131 may provide the prediction data PRD generated in this way to the multiple future state and probability predictor 132, and may output values related to the possible future states at the next time point (e.g., 20 hours later) and the possibility of reaching each state.

FIG. 12 is a block diagram illustrating a configuration of an apparatus 200 for non-deterministic future state prediction that includes only a preprocessor and a learner. In detail, the apparatus 200 of FIG. 12 may preprocess time series data and may train a prediction model for predicting the future state at an arbitrary time point based on the preprocessed time series data. The configuration and operation of a preprocessor 210 and a learner 220 of FIG. 12, and the configuration and operation of a learning database 201 and a model database 202 of FIG. 12 are the same as those described in FIG. 1.

FIG. 13 is a block diagram illustrating a configuration of an apparatus 300 for predicting a non-deterministic future state that includes only a predictor. In detail, the apparatus 300 of FIG. 13 may preprocess time series data that the user wants to predict and may use the predicted model to predict the future state at any point in time desired by the user. The configuration and operation of a preprocessor 310 and a predictor 320 in FIG. 13, and the configuration and operation of a user database 301, a model database 302, multiple future state prediction values 303, and future state probabilities 304 in FIG. 13 are the same as those described in FIG. 1.

In other words, according to an embodiment of the present disclosure, the operation of training a prediction model for predicting a non-deterministic future state and the operation of predicting a non-deterministic future state based on the trained prediction model may be performed individually through separate apparatuses (the apparatus 200 of FIG. 12 and the apparatus 300 of FIG. 13), or may be performed simultaneously through one apparatus (the apparatus 100 of FIG. 1).

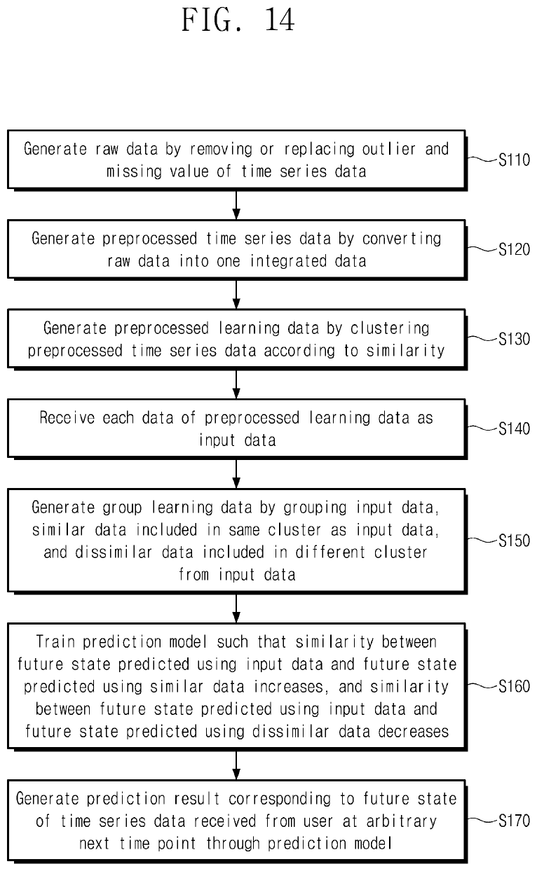

FIG. 14 is a flowchart illustrating a method of operating an apparatus for a non-deterministic future state prediction, according to an embodiment of the present disclosure. Hereinafter, it will be described with reference to FIGS. 1, 2 and 5 together with FIG. 14.

In operation S110, the outlier and missing value processor 111 may generate raw data by removing or replacing an outlier and a missing value of the time series data. In operation S120, the time series feature converter 112 may generate preprocessed time series data by converting the raw data into one integrated data. In operation S130, the similar state classifier 113 may generate preprocessed learning data by clustering the preprocessed time series data according to similarity.

In operation S140, the similar feature generator 121 may receive each data of the preprocessed learning data as input data, and in operation S150, may generate group learning data by grouping the input data, similar data included in the same cluster as the input data, and dissimilar data included in a different cluster from the input data. In operation S160, the learner 120 may train the prediction model such that the similarity between the future state predicted using the input data and the future state predicted using the similar data increases, and the similarity between the future state predicted using the input data and the future state predicted using the dissimilar data decreases.

In operation S170, the predictor 130 may generate a prediction result corresponding to the future state of the time series data received from the user at an arbitrary next time point through the prediction model.

Furthermore, the method for predicting a non-deterministic future state according to an embodiment of the present disclosure may be implemented as program code stored in a non-transitory computer-readable medium. For example, the non-transitory computer-readable media may include a magnetic media, an optical media, or combinations thereof (e.g., a CD-ROM, a hard drive, a read-only memory, a flash drive, etc.).

For example, the non-transitory computer-readable medium may include program code that, when executed, causes a processor to generate raw data by removing or replacing an outlier and a missing value in time series data, to generate preprocessed time series data by converting the raw data into one integrated data, to generate preprocessed learning data by clustering the preprocessed time series data depending on a similarity, to receive each data of the preprocessed learning data as input data, to generate group learning data by grouping the input data, similar data included in the same cluster as the input data, and dissimilar data included in a different cluster from the input data, to train a prediction model such that the similarity between a first future state predicted using the input data and a second future state predicted using the similar data increases and such that the similarity between the first future state and a third future state predicted using the dissimilar data decreases, and to generate a prediction result corresponding to a future state at an arbitrary next time point of the time series data received from a user through the prediction model.

According to an embodiment of the present disclosure discussed so far, non-deterministic future states that may appear differently depending on events may be predicted using time series data. The non-deterministic future state means that time series data with the same past record does not have a single definite future state, but may have various future states depending on additional events. Accordingly, a user (e.g., a medical professional) may plan a new event (e.g., a new treatment) by predicting the future state (e.g., the patient's condition) and probability of any next desired point in time.

Actually, data used in industrial environments, such as medical data, is often highly imbalanced or lacking in absolute quantity. According to an embodiment of the present disclosure, a prediction model may be robustly trained to accurately predict the future state at any point in time even when the data imbalance is severe or the absolute amount is insufficient. In addition, the embodiments so far have mainly been described with respect to time series medical data, but the present disclosure is not limited thereto, and methods for future prediction according to embodiments of the present disclosure may be applied to all types of time series data.

According to an embodiment of the present disclosure, non-deterministic future states that may appear differently depending on events may be predicted using time series data. This allows medical experts to plan new treatments by referring to various future states for patients.

In addition, according to an embodiment of the present disclosure, the future states may be accurately predicted through a robust model learning method with respect to imbalanced data such as medical data.

The above descriptions are specific embodiments for carrying out the present disclosure. Embodiments in which a design is changed simply or which are easily changed may be included in the present disclosure as well as an embodiment described above. In addition, technologies that are easily changed and implemented by using the above embodiments may be included in the present disclosure. While the present disclosure has been described with reference to embodiments thereof, it will be apparent to those of ordinary skill in the art that various changes and modifications may be made thereto without departing from the spirit and scope of the present disclosure as set forth in the following claims.

What is claimed is:

1. An apparatus comprising:
a preprocessor configured to generate raw data by removing or replacing an outlier and a missing value in time series data, to generate preprocessed time series data by converting the raw data into one integrated data, and to generate preprocessed learning data by clustering the preprocessed time series data depending on a similarity; and
a learner configured to receive each data of the preprocessed learning data as input data, and to train a prediction model such that a first similarity between a first future state predicted using the input data and a second future state predicted using data included in a same first cluster as the input data increases and such that a second similarity between the first future state and a third future state predicted using data included in a different second cluster from the input data decreases, and
wherein the prediction model is a machine learning model for predicting a future state of the time series data at an arbitrary time point.

2. The apparatus of claim 1, wherein the preprocessor is configured to generate the preprocessed time series data by adding a time interval between time points of the raw data as a next time point.

3. The apparatus of claim 1, wherein the learner includes:
a similar feature generator configured to generate group learning data by grouping the input data, similar data included in the same first cluster as the input data, and dissimilar data included in the different second cluster from the input data;
a multi-state calculator configured to calculate an input latent vector, a similar latent vector, and a dissimilar latent vector, respectively, representing changes over time in the input data, the similar data, and the dissimilar data;
a multi-state probability estimator configured to use the input latent vector to predict values associated with possible next states of the input data and probabilities of reaching each of the possible next states; and
a distance calculator configured to calculate a first similarity distance between the input latent vector and the similar latent vector and a second similarity distance between the input latent vector and the dissimilar latent vector.

4. The apparatus of claim 3, wherein the similar feature generator continuously generates the group learning data by changing the similar data to other similar data or changing the dissimilar data to different dissimilar data, with respect to the same input data while training the prediction model.

5. The apparatus of claim 3, wherein the multi-state calculator calculates the latent vectors using Equation 1,
xⁿᵢ₊₁=xᵢ+∫fₙ(xᵢ,tᵢ)dt+∫gₙ(xᵢ,tᵢ)dN (Equation 1)
where the xᵢ is an i-th record of the input data, the similar data, or the dissimilar data, the tᵢ is a next time point after an i-th time point of the input data, the similar data, or the dissimilar data, the fₙ(xᵢ, tᵢ) is a change rate estimation function, the ∫fₙ(xᵢ,tᵢ)dt is an amount of change in records from the i-th time point to the next time point, the gₙ(xᵢ, tᵢ) is a diffusion degree estimation function, the ∫gₙ(xᵢ,tᵢ)dN is an effect of random noise from the i-th time point to the next time point, and the latent vectors are a set of the xⁿᵢ₊₁.

6. The apparatus of claim 5, wherein the multi-state calculator includes a deep learning network for randomly calculating the change rate estimation function and the diffusion degree estimation function, and
wherein the deep learning network is one of a CNN (Convolutional Neural Network), an RNN (Recurrent Neural Network), or a BNN (Bayesian Neural Network).

7. The apparatus of claim 3, wherein the multi-state probability estimator calculates a loss function depending on Equation 2 based on the values associated with the possible next states and the probabilities, and trains the prediction model such that the loss function decreases,
L(y^{real},y,α)=−minpos(|y^{real}−y|)log(α) (Equation 2)
where the y^{real} is a value associated with an actual next state, the y is a value associated with a next state predicted by the input latent vector, and the α is the probability.

8. The apparatus of claim 7, wherein the multi-state probability estimator includes a fully connected layer that converts values associated with the possible next states into the corresponding probabilities.

9. The apparatus of claim 3, wherein the distance calculator calculates a loss function depending on Equation 3 based on the first similarity distance and the second similarity distance, and trains the prediction model such that the loss function decreases,
L(IV,SV,USV)=max(d(IV−SV)−d(IV−USV)+C,0) (Equation 3)
where the IV is the input latent vector, the SV is the similar latent vector, the USV is the dissimilar latent vector, the d(IV−SV) is the first similarity distance, the d(IV−USV) is the second similarity distance, and C is an arbitrary constant that may be specified in advance.

10. An apparatus comprising:
a preprocessor configured to generate raw data by removing or replacing an outlier and a missing value of time series data, and to convert the raw data into one integrated data to generate preprocessed time series data; and
a predictor configured to generate a prediction result corresponding to a future state at an arbitrary next time point in time through a prediction model, based on the preprocessed time series data, event data indicating an additional state not included in the preprocessed time series data, and next time point data indicating the arbitrary next time point, and
wherein the prediction model is a machine learning model for predicting a future state of the time series data at an arbitrary time point.

11. The apparatus of claim 10, wherein the preprocessor is configured to generate the preprocessed time series data by adding a time interval between time points of the raw data as a next time point.

12. The apparatus of claim 10, wherein the predictor includes:
a next time point reflector configured to generate prediction data by adding the event data to the preprocessed time series data and setting a next time point of the event data as the next time point data; and
a multiple future state and probability predictor configured to apply the prediction model to the prediction data and to output a value associated with a future state at a time point corresponding to the next time point data and a probability of reaching the future state.

13. A method of operating an apparatus for non-deterministic future state prediction using time series data, the method comprising:
generating raw data by removing or replacing an outlier and a missing value in time series data;
generating preprocessed time series data by converting the raw data into one integrated data;
generating preprocessed learning data by clustering the preprocessed time series data depending on a similarity;
receiving each data of the preprocessed learning data as input data;
generating group learning data by grouping the input data, similar data included in a same first cluster as the input data, and dissimilar data included in a different second cluster from the input data;
training a prediction model such that a first similarity between a first future state predicted using the input data and a second future state predicted using the similar data increases and such that a second similarity between the first future state and a third future state predicted using the dissimilar data decreases; and
generating a prediction result corresponding to a future state at an arbitrary next time point of the time series data received from a user through the prediction model.

14. The method of claim 13, wherein the training of the prediction model includes:
calculating an input latent vector, a similar latent vector, and a dissimilar latent vector, respectively, representing changes over time in the input data, the similar data, and the dissimilar data, depending on Equation 1,
xⁿᵢ₊₁=xᵢ+∫fₙ(xᵢ,tᵢ)dt+∫gₙ(xᵢ,tᵢ)dN (Equation 1)
where the xᵢ is an i-th record of the input data, the similar data, or the dissimilar data, the tᵢ is a next time point after an i-th time point of the input data, the similar data, or the dissimilar data, the fₙ(xᵢ, tᵢ) is a change rate estimation function, the ∫fₙ(xᵢ,tᵢ)dt is an amount of change in records from the i-th time point to the next time point, the gₙ(xᵢ, tᵢ) is a diffusion degree estimation function, the ∫gₙ(xᵢ,tᵢ)dN is an effect of random noise from the i-th time point to the next time point, and the latent vectors are a set of the xⁿᵢ₊₁.

15. The method of claim 14, wherein the training of the prediction model further includes:
predicting values associated with possible next states of the input data and probabilities of reaching each of the possible next states, by using the input latent vector; and
training the prediction model such that a loss function based on the values associated with the possible next states and the probabilities decreases, and
wherein the loss function is calculated according to Equation 2,
L(y^{real},y,α)=−minpos(|y^{real}−y|)log(α) (Equation 2)
where the y^{real} is a value associated with an actual next state, the y is a value associated with a next state predicted by the input latent vector, and the α is the probability.

16. The method of claim 14, wherein the training of the prediction model further includes:
calculating a first similarity distance between the input latent vector and the similar latent vector and a second similarity distance between the input latent vector and the dissimilar latent vector; and
training the prediction model such that a loss function based on the first similarity distance and the second similarity distance decreases,
wherein the loss function is calculated according to Equation 3,
L(IV,SV,USV)=max(d(IV−SV)−d(IV−USV)+C,0) (Equation 3)
where the IV is the input latent vector, the SV is the similar latent vector, the USV is the dissimilar latent vector, the d(IV−SV) is the first similarity distance, the d(IV−USV) is the second similarity distance, and C is an arbitrary constant that may be specified in advance.

17. The method of claim 13, wherein the generating of the prediction result includes:
generating second preprocessed time series data by removing or replacing an outlier and a missing value of time series data received from the user and converting the processed time series data into one integrated data;
receiving the second preprocessed time series data, event data indicating an additional state not included in the second preprocessed time series data, and next time point data indicating the arbitrary next time point;
generating prediction data by adding the event data to the second preprocessed time series data and setting a next time point of the event data as the next time point data; and
outputting a value associated with a future state at a time point corresponding to the next time point data and a probability of reaching the future state, by applying the prediction model to the prediction data.
